(19) United States
(12) Patent Application Publication
(10) Pub. No.: US 2022/0313967 A1
(43) Pub. Date: Oct. 6, 2022

(54) INGESTIBLE DEVICE WITH DELIVERY MEMBER DETACHMENT

(71) Applicant: Novo Nordisk A/S, Bagsvaerd (DK)

(72) Inventors: Morten Revsgaard Frederiksen, Copenhagen K (DK); Brian Jensen, Broenshoej (DK); Mikkel Oliver Jespersen, Copenhagen N (DK); Mette Poulsen, Glostrup (DK)

(21) Appl. No.: 17/616,386

(22) PCT Filed: Jun. 7, 2020
(86) PCT No.: PCT/EP2020/065755

§ 371 Date: Dec. 3, 2021

(30) Foreign Application Priority Data
Jun. 7, 2019 (EP) 19179205.0
Jun. 7, 2019 (EP) 19179206.8
Jul. 22, 2019 (EP) 19187663.0
Jan. 31, 2020 (EP) PCT/EP2020/052521

(51) Int. Cl. A61M 31/00; A61M 5/20; A61M 5/28; A61M 5/32

(52) U.S. Cl. CPC A61M 31/002; A61M 5/2033; A61M 5/286; A61M 5/3202; A61M 5/3287

(57) ABSTRACT

An ingestible device is disclosed suitable for swallowing into a lumen of a gastrointestinal tract of a patient, the lumen having a lumen wall. The ingestible device (100, 200) comprises: a capsule (110, 120), a delivery member (130) being shaped to penetrate tissue of a lumen wall and having a tissue penetrating end and a trailing end, the delivery member (130) comprising a therapeutic payload, a ram (150) attached relative to the delivery member (130) at the trailing end; and an actuator (140) coupled to the ram (150) and having a first configuration and a second configuration, the delivery member (130) being retained by the ram (150) within the capsule (110, 120) when the actuator (140) is in the first configuration, wherein the delivery member (130) is configured to be advanced from the capsule (110, 120) and into the lumen wall by movement of the actuator (140) from the first configuration to the second configuration such that the delivery member (130) moves along a predefined trajectory. The ram (150) is configured for being tilted relative to the predefined trajectory upon the actuator (140) moving into the second configuration to detach at least a portion of the delivery member (130) from the ram (150) such that the detached portion of the delivery member (130) remains within the lumen wall to release therapeutic payload.

The present invention relates to ingestible devices adapted for being swallowed into a lumen of a patient and having a delivery member being shaped to penetrate tissue of a lumen wall.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Prior art references relating to oral dosing of active agents and addressing one or more of the above challenges include WO 2018/213600 A1 and WO 2017/156347 A1.

Ingestible capsules have been proposed comprising a delivery member formed as a solid formed from a preparation comprising a therapeutic payload, wherein the delivery member is forced from the capsule and into tissue of the lumen wall for delivering the payload. The payload is inserted into tissue and will over time dissolve and be absorbed into the body of the patient. Even though the capsule may be able to properly orient relative to a target site it can still move to another location after deployment of the payload. This introduces the risk that the payload will be partly or fully removed from the target site due to movement of the capsule.

Having regard to the above, it is an object of the present invention to provide an ingestible device for swallowing into a lumen of a gastrointestinal tract, and which to a high degree effectively and reliably ensures proper deposition of the delivery member into tissue.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention an ingestible device is provided which is suitable for swallowing into a lumen of a gastrointestinal tract of a patient, the lumen having a lumen wall. The ingestible device comprises a capsule sized to be ingested by a patient; a delivery member disposable or disposed in the capsule, the delivery member being shaped to penetrate tissue of the lumen wall and having a tissue penetrating end and a trailing end opposite the tissue penetrating end. The delivery member comprises a therapeutic payload or is configured to deliver a therapeutic payload from a reservoir. The ingestible device further comprises a ram attached relative to the delivery member at the trailing end, and an actuator coupled to the ram and having a first configuration and a second configuration, the delivery member being retained by the ram within the capsule when the actuator is in the first configuration, wherein the delivery member is configured to be advanced from the capsule and into the lumen wall by movement of the actuator from the first configuration to the second configuration such that the delivery member moves along a predefined trajectory. The ram is configured for being tilted relative to the predefined trajectory upon the actuator moving into the second configuration to detach at least a portion of the delivery member from the ram such that the detached portion of the delivery member remains within the lumen wall to release therapeutic payload.

Due to the tilting movement of the ram relative to the delivery member which already has been inserted into tissue, a predefined portion, or the entire payload, is effectively detached from the ram, and thus effectively detached from the remaining part of the ingestible device. Hence, the risk that the capsule inadvertently provides a pulling force on the inserted delivery member is prevented.

In exemplary embodiments the delivery member is a solid formed partly or entirely from a preparation comprising the therapeutic payload, wherein the delivery member is made from a dissolvable material that dissolves when inserted into tissue of the lumen wall to deliver at least a portion of the therapeutic payload into tissue.

In other exemplary embodiments an exterior portion of the delivery member is made from a dissolvable solid material that dissolves when inserted into tissue of the lumen wall.

The exterior portion of the delivery member may be configured to define an enclosure, and wherein a preparation comprising the therapeutic payload forms a liquid, gel or powder accommodated within the enclosure.

In still further embodiments, the delivery member is an injection needle having a lumen, and wherein the therapeutic payload is provided as a liquid, gel or powder being expellable through the lumen of the injection needle from a reservoir within the capsule.

In some exemplary embodiments the delivery member forms an elongated member that, when the actuator assumes the first configuration, extends along a longitudinal axis, and wherein the predefined trajectory defines an axis coaxial with the longitudinal axis.

Alternatively, the delivery member may be provided in the form of an elongated member that extends along a curve, and wherein the predefined trajectory extends along said curve.

In some embodiments, the ingestible device comprises a tilt mechanism for tilting the ram as the actuator moves into the second configuration. In some embodiments, the ram cooperates with the capsule, or a structure associated with the capsule, to impose a tilting movement onto the ram as the actuator moves into the second configuration. In some embodiments, the actuator provides a force for tilting the ram. In other embodiments, in addition to said actuator, a separate actuator component is provided which imposes a tilting movement of the ram as the actuator moves into the second configuration.

In exemplary embodiments the capsule comprises a stop surface and the ram comprises a counter stop surface configured for engaging the stop surface of the capsule, wherein the stop surface of the capsule and the counter stop surface of the ram are formed to induce a tilting movement of the ram upon the actuator moving into the second configuration.

Some further embodiments provide a capsule that comprises a stop surface and the ram comprises a counter stop surface configured for engaging the stop surface of the capsule, wherein at least one of the stop surface and the counter stop surface comprises an eccentrically disposed protrusion, wherein the other of the stop surface and the counter stop surface is formed as a substantially planar surface, and wherein the protrusion and the planar surface induce a tilting movement of the ram upon the actuator moving into the second configuration.

In other embodiments, a guide system is arranged between the ram and the capsule, the guide system being configured to impose a tilting movement on the ram as the actuator moves into the second configuration.

In still other embodiments, a brake means arranged eccentrically to the ram is provided to impose a tilting movement on the ram as the actuator moves into the second configuration.

In still further embodiments, the ram may include a radially disposed bump or protrusion which serves to tilt the ram in the final part of the movement of the ram as the actuator moves into the second configuration.

In still further embodiments, the ram is configured to be tilted by cooperating with the actuator, wherein the actuator includes at least one member configured to act with an eccentrically disposed force component onto the ram.

The ram may in certain embodiments comprise an interface portion, wherein the trailing end of the delivery member attaches relative to the interface portion of the ram.

The trailing end of the delivery member may in some embodiments be attached relative to the interface portion of the ram by means of an adhesive.

In alternative embodiments, the trailing end of the delivery member attaches relative to the interface portion of the ram by means of one of a friction fit and a press fit.

In some embodiments the ram is configured upon firing to move the ram from a first position to a second position so that a major portion of the delivery member is inserted in tissue at a target location within the lumen wall, and wherein at least a portion of the delivery member is configured for detachment relative to the interface portion of the ram when the ram assumes the second position due to said tilting of the ram.

In some configurations the ram is movable from the first position to the second position by displacement in a pre-defined delivery stroke. In some embodiments, the tilting movement of the ram occurs only within the final 30% displacement of the delivery stroke, such as only within the final 20% displacement of the delivery stroke, such as only within the final 10% displacement of the delivery stroke, or such as within the final 5% displacement of the delivery stroke.

The actuator may comprise a drive spring, such as a compression spring, the spring being strained or configured for being strained for powering the ram.

In still other forms of the ingestible device the device is configured as a self-righting capsule, wherein when the self-righting capsule is at least partially supported by the tissue of the lumen wall, the self-righting capsule orients in a direction to allow the delivery member to be inserted into the lumen wall to deliver at least a portion of the therapeutic payload into the tissue.

The actuator may be provided as an energy source associated with the ram for powering the ram to expel or delivery the therapeutic payload. In some forms, the capsule and the ram comprises at least one pair of a latch and a retainer portion structured to maintain the ram in a pre-firing configuration. For each pair of latch and retainer portion the ingestible device defines a dissolvable firing member, the dissolvable firing member being at least partially dissolved in a fluid, such as a biological fluid, a retainer portion comprised by one of the capsule and the ram, and a deflectable latch comprised by the other of the capsule and the ram. The deflectable latch is configured for lateral movement relative to the axis, and the deflectable latch defines a first surface with a blocking portion, and a support surface disposed oppositely to the first surface and configured for interacting with the dissolvable firing member. In the pre-firing configuration, the blocking portion of the deflectable latch engages the retainer portion in a latching engagement, and the support surface of the deflectable latch interacts with the dissolvable firing member to restrict movement of the deflectable latch thereby preventing release of the latching engagement. In a firing configuration wherein the dissolvable firing member has become at least partially dissolved, the deflectable latch is allowed to move thereby releasing the latching engagement between the blocking portion of the deflectable latch and the retainer portion to allow the energy source to fire the ram.

By this arrangement, instead of having a dissolvable member that carries the whole power or load of the energy source, the dissolvable part is designed to simply block a mechanical activation system. The mechanical activation system may be designed to rely on parts made from a suitable high-strength material, such as plastic, and do not leave any undissolved pieces that potentially could jam the mechanical activation system.

In exemplary embodiments, the deflectable latch is configured for radial movement relative to the axis. In some examples the firing axis and the ram movement is linear. In other exemplary embodiments, the firing axis may be not linear, e.g. the firing trajectory of the ram may be arcuate or curved, or may include arcuate or curved trajectories. In accordance herewith, the latch may be configured for lateral movement relative to the trajectory of the ram to release the ram.

In exemplary embodiments a plurality of pairs of latch and retainer portions, such as two, three, four, five or more pairs of latch and retainer portions are provided, the pairs of latch and retainer portions being disposed equally around the axis.

In some embodiments said dissolvable firing member is common to all pairs of latch and retainer portions.

In further embodiments, the dissolvable firing member is arranged along the axis, wherein the at least one pair of latch and retainer portion is disposed radially outside of the dissolvable firing member.

In other variants one or more dissolvable firing members is/are disposed, such as in a ring-shaped configuration around the axis, wherein the one or more dissolvable firing members encircle the at least one pair of latch and retainer portion.

The capsule may comprise one or more openings to allow a biologic fluid, such as gastric fluid, to enter the capsule for dissolving the dissolvable firing member(s).

In some embodiments, the energy source is or comprises at least one spring configured as a drive spring. Exemplary springs include a compression spring, a torsion spring, a leaf spring or a constant-force spring. The spring may either be strained or configured for being strained for powering the ram. Other non-limiting exemplary types of energy sources for the actuator include compressed gas actuators or gas generators. In some embodiments, in the pre-firing configuration, the energy source exerts a load onto the ram thereby biasing the ram along the axis. In other embodiments the energy source is configured to exert a load onto the ram only upon triggering of a trigger member or mechanism of the ingestible device.

In exemplary embodiments, the ingestible device is configured for swallowing by a patient and travelling into a lumen of a gastrointestinal tract of a patient, such as the stomach, the small intestines or the large intestines. The capsule of the device may be shaped and sized to allow it to be swallowed by a subject, such as a human.

In still further exemplary embodiments, the ingestible device is configured as a self-righting capsule, wherein when the self-righting capsule is at least partially supported by the tissue of the lumen wall, the self-righting capsule orients in a direction to allow the delivery member to be inserted into the lumen wall to deliver at least a portion of the therapeutic payload into the tissue. The ingestible device may in certain embodiments be configured as a self-righting capsule device having a geometric center and a center of mass offset from the geometric center along the axis, wherein when the capsule device is supported by the tissue of the lumen wall while being oriented so that the centre of mass is offset laterally from the geometric center the capsule device experiences an externally applied torque due to gravity acting to orient the capsule device with the axis oriented along the direction of gravity to enable the delivery member to interact with the lumen wall at the target location.

By the above arrangements an orally administered drug substance can be delivered safely and reliably into the stomach wall or intestinal wall of a living mammal subject. The drug substance may e.g. be in the form of a solid, an encapsulated solid, a liquid, a gel or a powder, or any combination thereof.

As used herein, the terms “drug”, “drug substance” or “payload” is meant to encompass any drug formulation capable of being delivered into or onto the specified target site. The drug may be a single drug compound or a premixed or co-formulated multiple drug compound. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIGS. 1a and 1b each shows a cross-sectional front view of a first embodiment of a capsule device in accordance with the invention configured for solid dose delivery, the device assuming a pre-firing configuration and a firing configuration, respectively,

FIG. 2 shows schematically three different configurations of an assembly of a ram and a solid dose delivery member for use in a capsule device according to an aspect of the invention,

FIG. 3 shows schematically four different configurations of pairs of deformable latch and retaining portion assemblies for use in firing a ram in a capsule device according to the invention,

FIG. 4 shows schematically three different configurations of a capsule and ram assembly to enable solid dose delivery detachment between a solid delivery member and a ram,

FIGS. 5a and 5b each schematically shows a cross-sectional front view of a second embodiment of a capsule device in accordance with the invention configured for solid dose delivery, the device assuming a pre-firing configuration and a firing configuration, respectively, and

FIGS. 6a and 6b each schematically shows a cross-sectional front view of a third embodiment of a capsule device in accordance with the invention configured for solid dose delivery, the device assuming a pre-firing configuration and a firing configuration, respectively.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

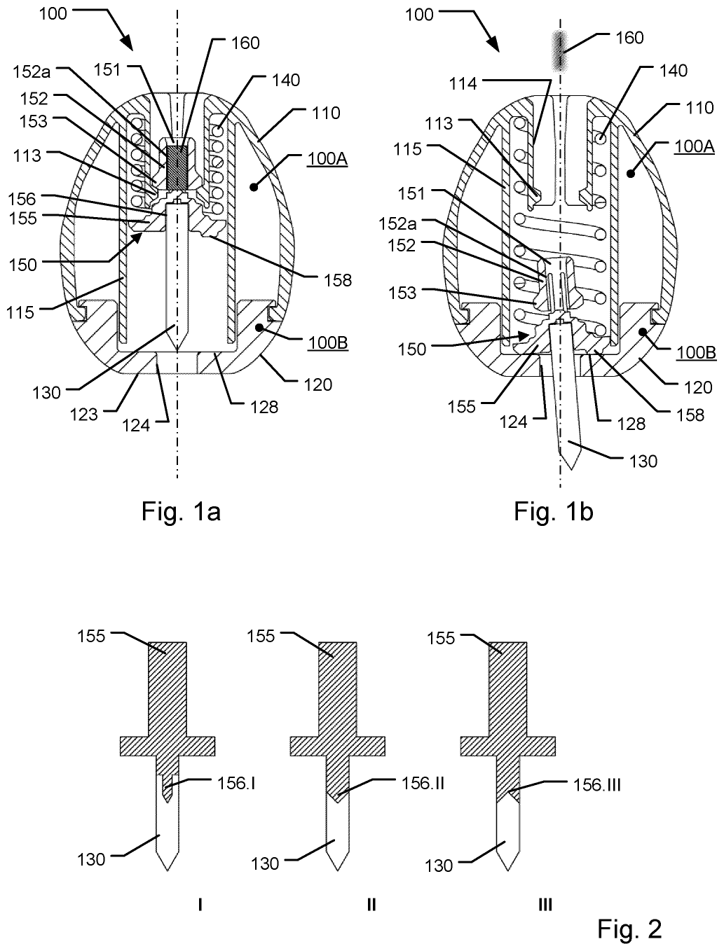

With reference to FIGS. 1a and 1b a first embodiment of a drug delivery device in accordance with an aspect of the invention will be described, the embodiment being designed to provide a capsule device 100 having a desired firing principle for deployment of a solid dose from a solid dose capsule device. It is to be noted that the disclosed firing principle is only exemplary and, in accordance with the invention, other firing principles may be used in alternative embodiments. The disclosed embodiment relates to a capsule device 100 suitable for being ingested by a patient to allow the capsule device to enter the stomach lumen, subsequently to orient relative to the stomach wall, and finally to deploy a solid dose payload for insertion at a target location in tissue of the stomach wall. For the capsule device 100 the general principle for orienting the capsule relative to the stomach wall may utilize any of the principles disclosed in WO 2018/213600 A1.

The ingestible self-righting capsule device 100 comprises a first portion 100A having an average density, a second portion 100B having an average density different from the average density of the first portion 100A. The capsule device 100 accommodates a payload portion 130 for carrying an agent for release internally of a subject user that ingests the article. In the shown embodiment, the average density of capsule device prior to deployment is larger than that of gastrointestinal fluid, enabling the capsule device to sink to the bottom of the stomach lumen. The outer shape of the self-righting article is a gomboc shape, i.e. a gomboc-type shape that, when placed on a surface in any orientation other than a single stable orientation of the shape, then the shape will tend to reorient to its single stable orientation.

The capsule device shown includes an upper (proximal) capsule part 110 which mates and attaches to a lower (distal) capsule part 120. The upper capsule part 110 and the lower capsule part 120 together forms the capsule of the device. The capsule defines an interior hollow which accommodates the payload portion 130, a ram 150 which holds and drives forward the payload portion 130, and a firing and propulsion mechanism including an actuator configured to fire and drive forward the ram with the payload for drug delivery. The payload portion 130 is oriented along a firing axis and configured for movement along the firing axis. In the shown embodiment, the upper and lower capsule parts 110, 120 form rotation symmetric parts which are symmetric around the firing axis. In the drawings, the device is oriented with the firing axis pointing vertically, and with the payload portion 130 pointing vertically downwards towards an exit hole 124 arranged centrally in the lower capsule part 120, the exit hole allowing the payload portion 130 to be transported through exit hole and moved outside the capsule device 100. The lower part 120 includes a tissue engaging surface 123 which is formed as a substantially flat lower outer surface surrounding the exit hole 124.

Regarding suitable materials for the capsule parts for the embodiment shown in FIGS. 1a and 1b, the upper part may suitably be made from a low-density material, such as polycaprolactone (PCL), whereas the lower part 120 may be suitably made from a high-density material, such as 316L stainless steel.

In the shown embodiment, due to the density distribution of the entire capsule device 100, and due to the outside shape of the device, the capsule device 100 will tend to orient itself with the firing axis substantially perpendicular to the surface (e.g., a surface substantially orthogonal to the force of gravity, a surface of a tissue such as the wall of the gastrointestinal tract). Hence, the capsule device tends to orient relative to the direction of gravity so that the tissue engaging surface 123 faces vertically downward.

The interior of the upper capsule part 110 includes a sleeve shaped ram guiding structure 115 which extends concentrically with the firing axis from the upper part of the upper capsule part 110 towards a ram stop surface 128 defined by an inner bottom surface formed in the lower capsule part 120, i.e. a proximally facing stop surface. Further, in the shown embodiment, a second sleeve shaped structure 114 extends concentrically with the firing axis and radially inside the ram guiding structure 115 from the upper capsule part 110 and downwards along the firing axis. The second sleeve shaped structure 114 serves as a retainer structure for retaining the ram 150 against the drive force emanating from a strained drive spring 140 arranged within the capsule, i.e. the drive spring serves as an actuator for driving forward the ram from a first position to a second position. In the shown embodiment, the retainer structure has a radially inwards protruding retainer portion 113 arranged at the lower end of the retainer structure. In the shown embodiment, the retainer portion 113 is provided as two opposed radially inwards protruding arc-shaped protrusions.

In the first embodiment shown in FIGS. 1a and 1b, payload portion 130 defines a solid delivery member formed entirely or partly from a preparation comprising the therapeutic payload. In the shown embodiment, the solid delivery member is formed as a thin cylindrical rod shaped to penetrate tissue of the lumen wall, the cylindrical rod having a tissue penetrating end and trailing end opposite the tissue penetrating end. The tissue penetrating end of the rod is pointed to facilitate easy insertion into tissue of the lumen wall whereas the trailing end, in the shown embodiment, defines a truncated cylinder cut off by a 90-degree cut. A non-limiting example of a drug suitable for delivery by capsule device 100 is dried compressed API such as insulin.

The ram 150 comprises an upper retaining part 151 and a lower interface part 155 configured for holding the trailing end of the payload portion 130 in place. In the shown embodiment, the interface part includes a downward open bore that receives the trailing end of the payload portion 130 in a way so that the payload portion 130 is firmly attached within the bore. The lower interface part 155 further defines an annular outer flange having a diameter slightly smaller than the diameter of the ram guiding structure 115. In the shown embodiment, the ram 150 is movable, while being guided for axial movement by the ram guiding structure 115, from a pre-firing configuration shown in FIG. 1a to a firing configuration shown in FIG. 1b.

With regard to the above-mentioned drive spring 140, in capsule device 100, a helical compression spring is arranged coaxially with the firing axis. The proximal end of drive spring 140 is seated against a spring seat of upper capsule part 110, i.e. located radially between the ram guiding structure 115 and the retainer structure. The distal end of drive spring 140 is seated against a spring seat formed by a proximal surface of the flange defined by the lower interface part 155 of the ram 150. As part of assembling the capsule device 100 the drive spring 140 has been energized by axially compressing the drive spring 140 between the two spring seats. Hence, the ram is initially under load from drive spring, such as in the order of 10-30 N. Alternatives to using a compression spring for generating the drive force, other spring configurations may be used to energize the capsule device 100, such as a torsion spring, a leaf spring, a constant-force spring or similar. In further alternatives, a gas spring or a gas generator may be used.

The upper retaining part 151 of the ram 150 includes deflectable latches provided in the form of two deflectable arms 152 which extend in distal direction from the upper end of the ram towards the exit opening 124, each arm being resiliently deflectable in the radial inwards direction. The end of each deflectable arm 152 includes a blocking portion 153 protruding radially outwards from the resilient arm. In the pre-firing configuration shown in FIG. 1a, a distal surface of each of the blocking portions 153 engage a proximal surface of a corresponding one the retainer portions 113. As the blocking portions 153 initially are located proximally to the retainer portions 113 the ram 150 cannot be moved distally past the retainer portions 113 unless the deflectable arms 152 become sufficiently deflected in the radially inwards direction.

In the pre-firing configuration a dissolvable pellet 160 is arranged between the two deflectable arms 152 so that radial opposing surfaces of the pellet 160 engage a radially inwards facing support surface of the two deflectable arms 152. In the shown embodiment, the pellet 160 is arranged in a compartment inside the upper capsule part 110, and a proximally arranged upper opening in upper capsule part 110 facilitates fluid exposure to the dissolvable pellet when the capsule device is submerged in a fluid. In the pre-firing configuration shown in FIG. 1a, as the dissolvable pellet 160 assumes a non-compressible state the pellet prevents the two deflectable arms from bending inwards. However, upon exposure to a fluid, such as gastric fluid present in the stomach of a patient, the dissolvable pellet starts to dissolve. The pellet 160 is designed to become gradually dissolved so that after a predefined activation time, the pellet has been dissolved to a degree allowing the two deflectable arms 152 to become sufficiently deflected inwards enabling the blocking portions 153 of ram 150 to be moved distally past the retainer portions 113. In this condition, i.e. the firing configuration, the ram 150 has been fired with the load of the drive spring 140 forcing the ram 150 distally towards the exit hole 124. The ram 150 drives the payload portion 130 distally with the payload tip protruding initially from the capsule, and gradually pressing out the remaining payload portion 130. The forward movement of the payload portion 130 is halted when ram 150 bottoms out in the lower capsule part 120. This condition is depicted in FIG. 1b.

In the shown embodiment, the interface between the retainer portions 113 and the blocking portions 153 is sloped by approximately 30° so that the deflectable arms will slide inwards when the dissolvable pellet is dissolved. The angle determines the shear forces on the pellet and to which degree the deflectable arms will tend to slide inwards when subjected to the load force. In connection with the acceleration length of the ram when fired, the optimal angle is 0°, but it requires a much higher spring force to activate such configuration. For the sloped portions, in other embodiments, angles other than 30° may be used.

FIG. 1b reveals that, in the shown embodiment, the ram 150 and the payload portion 130 may enter an orientation that is somewhat tilted relative to the firing axis. This effect is obtained by a tilting mechanism that tilts the ram 150 upon the ram reaching its end destination, i.e. the end of stroke position. However, the condition schematically shown in FIG. 1b is somewhat hypothetical, as it is only representative for a capsule device being fired into the open, or with the payload portion being fired into a fluid.

In situation of intended use, the payload portion 130 is inserted into tissue of the lumen wall where it will anchor generally in a direction along the firing axis. However, at the end of the drive stroke, and due to the tilting action of the ram 150, a bending torque is applied onto payload portion 130 tending to break or otherwise release the connection between payload 130 and ram 150. This effect is introduced to enable the payload portion 130 to become forcedly separated from the ram 150 to prevent that payload portion 130 becomes withdrawn from the tissue after it has been properly lodged within the tissue.

At this point the capsule device 100 has delivered the intended dose and will release relative to the deposited payload portion 130 which rests inside the tissue wall. Subsequently, the remaining parts of the capsule device will travel out through the digestive system of the user and be disposed of.

If the payload 130 where still fixedly connected to ram 150, and thus also to the remaining parts of the capsule device 100, the likelihood that payload portion would become retracted from the tissue by movements of the capsule device relative to the target location would be high.

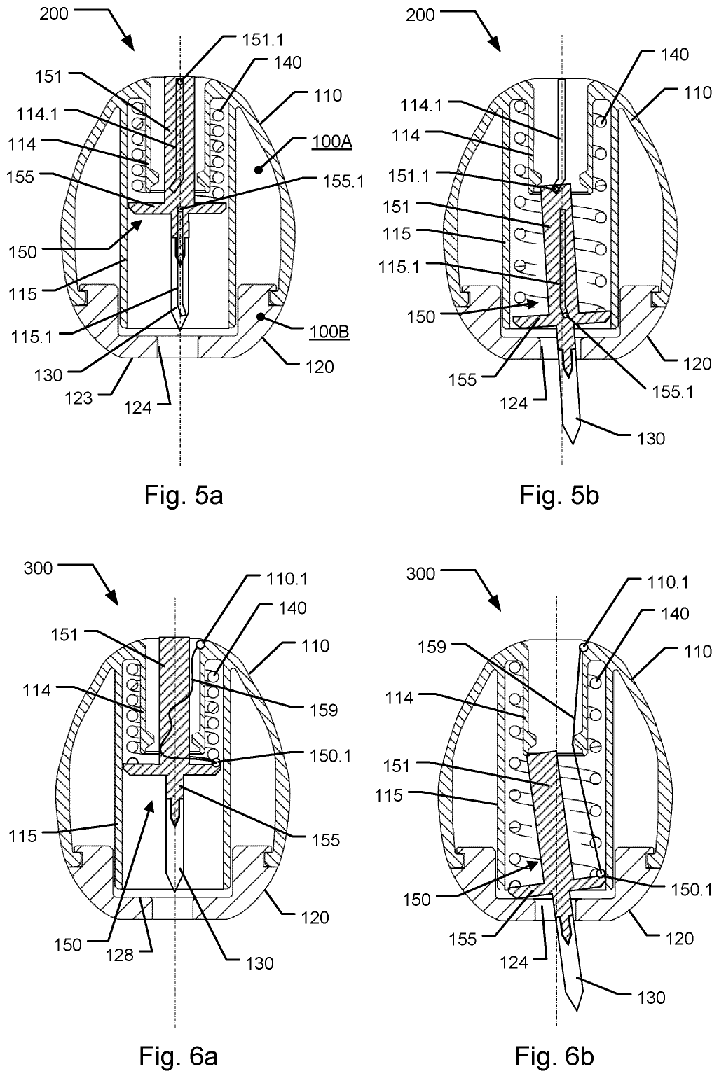

In the shown embodiment, the tilting motion of ram 150 upon reaching the end destination is obtained by forming an eccentrically arranged protrusion 158 on the distally facing surface of interface part 155 of ram 150. As proximally facing ram stop surface 128 defined by the inner bottom surface formed in the lower capsule part 120 is planar, and oriented orthogonally to the firing axis, a tilting effect is obtained as ram 150 meets the ram stop surface 128. As will be discussed further below, the tilting effect may be obtained by a variety of alternative geometrical designs. Also, as shown in connection with FIGS. 5a and 5b, a guide system between the capsule parts and the ram, such as between the guiding structure 115 and the ram 150, may alternatively be formed to obtain a similar tilting effect.

For the dissolvable member discussed above, i.e. the dissolvable pellet 160 forming a dissolvable firing member, different forms and compositions may be used. Non-limiting examples include injection moulded Isomalt pellets, compressed granulate Isomalt pellets, compressed pellets made from a granulate composition of Citrate/NaHCO3, or compressed pellets made from a granulate composition of Isomalt/Citrate/NaHCO3. A non-limiting exemplary size of a dissolvable pellet is a pellet which at the time of manufacturing measures Ø1×3 mm.

In the shown example of ram 150 the upper retaining part 151 is formed as a chamber wherein the dissolvable pellet 160 is received within the chamber having a tight fit. In the shown embodiment, the central upper part of capsule device 100 includes a single opening for introducing stomach fluid within the capsule. In other embodiments, the capsule may include other design of fluid inlet openings such as multiple openings distributed around the capsule. In some designs, the payload portion 130 is accommodated in a chamber that is fluidly sealed from the chamber of the dissolvable pellet. Also, the exit hole 124 may include a seal preventing moisture from entering the payload portion chamber prior to firing of the capsule device 100.

Turning now to FIG. 2, three alternative suitable designs for a ram and payload portion are schematically depicted, each design obtaining a desired attachment between ram 150 and payload portion 130 and enabling a desired controlled detachment of payload portion 130 from ram 150.

Design no. 1 includes a ram 150 having a central pin 156.I extending from lower interface part 155 of the ram 150. Payload portion 130 is correspondingly formed with a central opening configured for receiving central pin 156.I.

Design no. 11 includes a ram 150 having a central conical protrusion 156.II extending from lower interface part 155 of the ram 150. Payload portion 130 is correspondingly formed with a central conical depression configured for mating with and receiving conical protrusion 156.II.

Design no. III includes a ram 150 having a central conical depression 156.III at the distal facing surface of lower interface part 155 of ram 150. Payload portion 130 is correspondingly formed with a central conical protrusion configured for mating with and receiving conical protrusion 156.III.

The above described four different variants of interfaces between the payload portion 130 and the ram 150 are only exemplary and other configurations may be used instead. The detachable attachment between the payload portion and the ram may be obtained by using a friction or press fit. Alternatively, an adhesive may be used at the interface, such as sucrose. Still alternatively, the attachment may be obtained by initially wetting the payload portion and utilizing inherent stiction between the ram and the payload portion. In situation of use, upon the ram reaching its final destination, detachment may occur at the interface between the payload portion and the ram. In other embodiments, a desired detachment may be obtained by detaching a major portion of the payload portion from the remaining payload portion being still adhered or fastened to the ram. In some embodiments, the payload portion includes a weakened point which determines the point of separation. In still further embodiments, as discussed further below, the ram and the payload portion may be formed as a unitary component all made of a composition containing API, and wherein the intended payload portion to be pushed out from capsule device is separated from the ram portion.

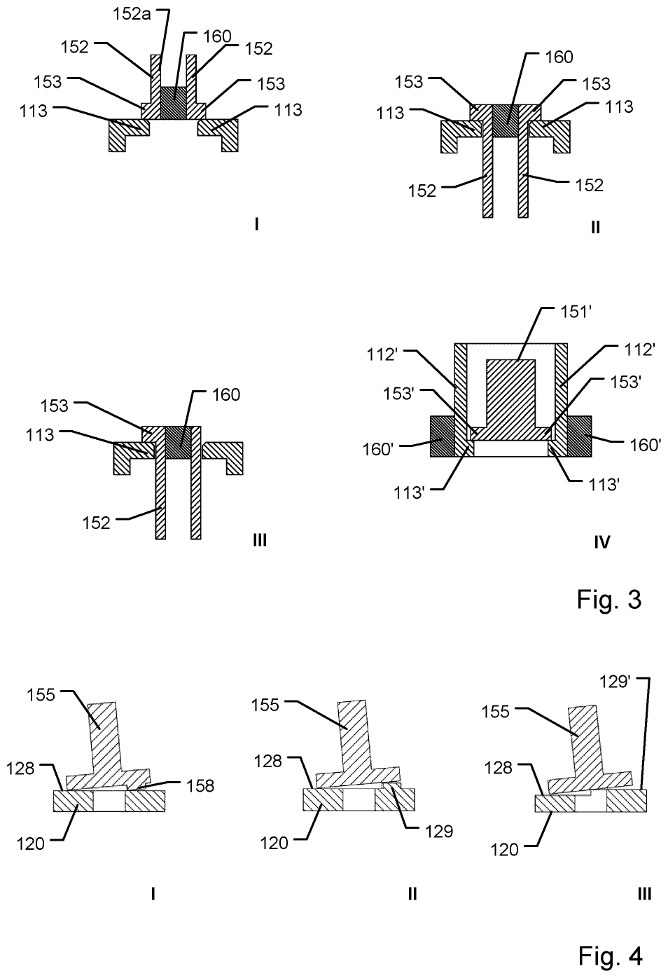

FIG. 3 schematically shows four additional designs for one or two pairs of deflectable latch and retainer configurations to be used in further exemplary capsule devices. As will be readily apparent, the number of deflectable latch elements, the location and the orientation of deflectable latch elements, the number and configuration of dissolvable firing members as well as the design of the ram may be varied in agreement with an aspect of the present invention while still obtaining firing mechanisms having a superior mode of action. For simplicity, only the upper retaining part 151 of ram 150 has been shown. Likewise, only the retainer structure of the capsule parts have been shown.

In FIG. 3, design no. I a retainer portion having upwardly extending retaining structure 113 to cooperate with blocking elements on two deflectable arms 152 is shown. In this design, a ram and a dissolvable firing member 160 having an overall structure as shown in FIG. 1a may be used.

Design no. II also includes an upwardly extending retaining structure 113 wherein a major portion of the ram is suspended. In this embodiment, the ram includes proximally extending delectable arms having blocking elements on the proximal ends of the deflectable arms 152, and wherein the proximal ends of the arms are designed to flex radially inwards when a centrally located dissolvable firing member 160 is sufficiently dissolved.

The figure depicting design no. III shows a related configuration but wherein the ram only includes a single deflectable arm. In this design a non-deflectable structure is arranged on the side of the dissolvable firing member 160 on the side facing away from the single deflectable arm. The non-deflectable structure continuously supports the dissolvable firing member 160 on one side thereof whereas the opposing side makes room for the single deflectable latch arm to move radially inwards and pass the retainer portion 113.

Finally, design no. IV schematically shows an example wherein the deflectable latch and the retainer portions have swapped places. In this design the ram includes an upper retaining portion 151′ with retainer portions 153′ which are designed not to exhibit any flexure during firing of the actuation mechanism. The retaining structure (associated with either the upper capsule part or the lower capsule part) instead includes two deflectable latches in the form of distally extending deflectable latch arms 112′, each having a blocking portion 153′ at its most distal end. Each deflectable arm 112′ is configured to engage a respective dissolvable firing member 160′. Said respective dissolvable firing members 160′ may thus be provided as a common ring-shaped member or be provided as a plurality of separate members arranged in a ring-configuration around the firing axis. As noted above, in some embodiments, the payload may act as a ram by itself to be partly or fully disconnected from the remainder of the capsule device. Such API based ram may include retainer portions which are designed not to exhibit any flexure during firing of the actuation mechanism where the retainer portions are allowed to pass cooperating deflectable latches associated with the housing of the capsule, e.g. the upper or lower capsule parts.

FIG. 4 schematically shows three designs for obtaining the tilting effect of the ram 150 as described above. In design no. I, an eccentrically disposed protrusion 158 is formed on the distally facing surface of interface part 155 of ram 150, i.e. the surface facing the ram stop surface 128. In design no. II, an eccentrically disposed protrusion 129 on the ram stop surface 128 is located to protrude in the proximal direction towards the lower surface of the interface part 155 of ram 150. In the variant shown as design III the ram stop surface 128 is formed as a stepped surface 129′, i.e. comprising two or more levels that induces a tilting movement of ram 150 as it reaches the ram stop surface 128. It is to be noted that other ways of tilting the ram upon reaching the final destination than shown schematically in FIG. 4 may be carried out by other means.

With reference to FIGS. 5a-5b, a second embodiment of a drug delivery device in accordance with the invention is schematically shown, the second embodiment providing a capsule device 200 being designed with a tilting mechanism which provides an alternative to the tilting mechanism described in connection with FIGS. 1a-1b, and 4.

With regard to the self-righting ability and the firing principle, the second embodiment capsule device 200 generally corresponds to the overall design of the first embodiment 100, but the way the ram is moved from the first position to the second position is different. In the shown second embodiment, the ram 150 is movable, while being guided for movement by a system of track and track followers, from a pre-firing configuration shown in FIG. 5a to a firing configuration shown in FIG. 5b. In the drawings details which mainly relate to the firing principle have been omitted for simplicity.

The interior of the upper capsule part 110 again includes a sleeve shaped ram guiding structure 115 which extends concentrically with the firing axis from the upper part of the upper capsule part 110 towards the lower capsule part 120. Further, in the second embodiment, a second sleeve shaped structure 114 extends concentrically with the firing axis and radially inside the ram guiding structure 115 from the upper capsule part 110 and downwards along the firing axis. The second sleeve shaped structure again serves as a retainer structure for retaining the ram 150 against the drive force emanating from strained drive spring 140 arranged within the capsule, i.e. the drive spring serves as an actuator for driving forward the ram from a first position to a second position. In addition, in this embodiment, the second sleeve shaped structure 114 serves as an additional guide for guiding the ram during its movement from the first position to the second position. A first pair of opposed guiding tracks 115.I are formed in the ram guiding structure 115 whereas a second pair of opposed guiding tracks 114.I are formed in the second sleeve shaped structure 114. Each pair of opposed first and second guiding tracks includes a relatively long axially extending segment which extends in parallel with the firing axis and includes a relatively short angled segment being inclined with respect to the firing axis.

The ram 150 again comprises an upper retaining part 151 and a lower interface part 155 configured for holding the trailing end of the payload portion 130 in place. The lower interface part 155 defines an annular outer flange having a diameter slightly smaller than the diameter of the ram guiding structure 115. In the shown embodiment, the said guiding tracks and cooperating track followers define the movement of the ram 150 as it moves from the pre-firing configuration shown in FIG. 5a to the firing configuration shown in FIG. 5b.

In the second embodiment capsule device 200, the flange of lower interface part 155 has two opposed track followers, each track follower being provided as a guide pin 155.I arranged to be guided by a respective one of the first pair of opposed guiding tracks 115.I. In addition, the upper retaining part 151 of the ram has two opposed track followers, each track follower being provided as a guide pin 151.I arranged to be guided by a respective one of the second pair of opposed guiding tracks 114.I.

When the capsule device is in the initial pre-firing configuration, cf. FIG. 5a, the guide pins 155.I are located in the axial segments of the guiding tracks 115.I, whereas the guide pins 151.I are located in the axial segments of the guiding tracks 114.I. Upon firing of the capsule device 200, after ingestion, the ram 150 moves axially along the firing axis for a substantial part of the entire stroke that the ram is designed to experience. During this part of movement the ram 150 drives the payload portion 130 distally with the payload tip protruding initially from the capsule, and gradually pressing out the remaining payload portion 130.

Shortly before the ram reaches the end of stroke position, i.e. shortly before the ram assumes the position shown in FIG. 5b, the guide pins 155.I and 151.I reaches the inclined segments of the guiding tracks 115.I and 114.I. Due to the inclination direction of the guiding tracks the lower interface part 155 of the ram is moved laterally in a first direction (to the right in FIG. 5b), whereas the upper retaining part 151 of the ram is moved laterally in a second direction opposite to the first direction (to the left in FIG. 5b). This induces a tilting movement of the ram shortly before it reaches the end of stroke position shown.

Similarly to what has been noted previously, the condition schematically shown in FIG. 5b is somewhat hypothetical, as it is only representative for a capsule device being fired into the open, or with the payload portion being fired into a fluid. In situation of intended use, the payload portion 130 is inserted into tissue of the lumen wall where it will anchor generally in a direction along the firing axis. However, at the end of the drive stroke, and due to the tilting action of the ram 150, a bending torque is applied onto payload portion 130 tending to break or otherwise release the connection between payload 130 and ram 150. This effect is introduced to enable the payload portion 130 to become forcedly separated from the ram 150 to prevent that payload portion 130 becomes withdrawn from the tissue after it has been properly lodged within the tissue. In other embodiments, in accordance with an aspect of the invention, different track and track follower configurations can be envisioned to provide the desired tilting movement of the ram upon

A further alternative tilting mechanism is schematically shown in FIGS. 6a and 6b which depict a third embodiment of a capsule device 300 in accordance with the invention. In this embodiment the tilting mechanism is provided by means of a wire or string 159 mounted between the capsule and the ram and having sufficient length to allow the ram to move for deployment of the payload portion into tissue, but which halts a portion of the ram at a desired end of stroke position to induce a tilting motion of the ram shortly before the end of stroke position. A first end of the string 159 is anchored eccentrically at a first anchor point 150.I on the flange of the lower interface part 155 of ram 150. A second end of the string 159 is anchored at a second anchor point 110.I disposed at the upper capsule part 110.

As indicated in FIG. 6a, when the capsule device 300 assumes the pre-firing configuration, the string 159 is arranged loosely between the first anchor point 150.I and the second anchor point 110.I inside the upper capsule part 110. Upon firing of the capsule device 300, after ingestion, the ram 150 moves axially along the firing axis for a substantial part of the entire stroke that the ram is designed to experience. During this part of movement the ram 150 drives the payload portion 130 distally with the payload tip protruding initially from the capsule, and gradually pressing out the remaining payload portion 130 into tissue.

Shortly before the ram reaches the end of stroke position, i.e. shortly before the ram assumes the position shown in FIG. 6b, the string 159 becomes stretched between the anchor points 150.I and 110.I thereby halting the side of the ram where the anchor point 150.I is located. The side of the ram located diametrically opposite to the anchor point 150.I is forced further by the drive spring 140 for slight distal movement until the flange of lower interface part 155 bottoms out relative to ram stop surface 128. This induces a tilting movement of the ram 150 shortly before it reaches the end of stroke position shown. Hence, at the end of the drive stroke, and due to the tilting action of the ram 150, a bending torque is applied onto payload portion 130 causing it to break or otherwise release the connection between payload 130 and ram 150.

Although the above description of exemplary embodiments mainly concern ingestible capsules for delivery in the stomach, the present deployment principle generally finds utility in capsule devices for lumen insertion in general, wherein a capsule device is positioned into a body lumen for deployment of a delivery member. Non-limiting examples of capsule devices may include capsule devices for intestinal delivery of a drug by delivery into the tissue wall of an intestinal lumen, such as a lumen of the small intestines or a lumen of the large intestines. Drug delivery may be performed using a delivery member, such as a needle, or via microneedles which is/are inserted into the tissue wall, such as wherein a microneedle array is becoming detached relative to a ram.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An ingestible device suitable for swallowing into a lumen of a gastrointestinal tract of a patient, the lumen having a lumen wall, the ingestible device comprising:
a capsule sized to be ingested by a patient;
a delivery member disposable in the capsule, the delivery member being shaped to penetrate tissue of the lumen wall and having a tissue penetrating end and a trailing end opposite the tissue penetrating end, the delivery member comprising a therapeutic payload or being configured to deliver a therapeutic payload from a reservoir;
a ram attached relative to the delivery member at the trailing end; and
an actuator coupled to the ram and having a first configuration and a second configuration, the delivery member being retained by the ram within the capsule when the actuator is in the first configuration, wherein the delivery member is configured to be advanced from the capsule and into the lumen wall by movement of the actuator from the first configuration to the second configuration such that the delivery member moves along a predefined trajectory, wherein the ram is configured for being tilted relative to the predefined trajectory upon the actuator moving into the second configuration to detach at least a portion of the delivery member from the ram such that the detached portion of the delivery member remains within the lumen wall to release therapeutic payload.

2. The ingestible device as in claim 1, wherein the delivery member is a solid formed entirely from a preparation comprising the therapeutic payload, wherein the delivery member is made from a dissolvable material that dissolves when inserted into tissue of the lumen wall to deliver at least a portion of the therapeutic payload into tissue.

3. The ingestible device as in claim 1, wherein an exterior portion of the delivery member is made from a dissolvable solid material that dissolves when inserted into tissue of the lumen wall.

4. The ingestible device as in claim 3, wherein the exterior portion of the delivery member defines an enclosure, and wherein a preparation comprising the therapeutic payload forms a liquid, gel or powder accommodated within the enclosure.

5. The ingestible device as in claim 3, wherein the delivery member is an injection needle, and wherein the therapeutic payload is provided as a liquid, gel or powder being expellable through the injection needle from a reservoir within the capsule.

6. The ingestible device as in claim 1, wherein the delivery member forms an elongated member that, when the actuator assumes the first configuration, extends along a longitudinal axis, and wherein the predefined trajectory defines an axis coaxial with the longitudinal axis.

7. The ingestible device as in claim 1, wherein the delivery member forms an elongated member that extends along a curve, and wherein the predefined trajectory extends along said curve.

8. The ingestible device as in claim 1, wherein the capsule comprises a stop surface and the ram comprises a counter stop surface configured for engaging the stop surface of the capsule, wherein the stop surface of the capsule and the counter stop surface of the ram are formed to induce a tilting movement of the ram upon the actuator moving into the second configuration.

9. The ingestible device as in claim 1, wherein the capsule comprises a stop surface and the ram comprises a counter stop surface configured for engaging the stop surface of the capsule, wherein at least one of the stop surface and the counter stop surface comprises an eccentrically disposed protrusion, wherein the other of the stop surface and the counter stop surface is formed as a substantially planar surface, and wherein the protrusion and the planar surface induce a tilting movement of the ram upon the actuator moving into the second configuration.

10. The ingestible device as in claim 1, wherein the ram comprises an interface portion and wherein the trailing end of the delivery member attaches relative to the interface portion of the ram.

11. The ingestible device as in claim 10, wherein the trailing end of the delivery member attaches relative to the interface portion of the ram by structure of an adhesive.

12. The ingestible device as in claim 10, wherein the trailing end of the delivery member attaches relative to the interface portion of the ram by structure of one of a friction fit and a press fit.

13. The ingestible device as in claim 10, wherein the ram is configured upon firing to move the ram from a first position to a second position so that a major portion of the delivery member is inserted in tissue at a target location within the lumen wall, and wherein at least a portion of the delivery member is configured for detachment relative to the interface portion of the ram when the ram assumes the second position due to said tilting of the ram.

14. The ingestible device as in claim 1, wherein the actuator comprises a drive spring, such as a compression spring, the spring being strained or configured for being strained for powering the ram.

15. The ingestible device as in any of claim 1, wherein the ingestible device is configured as a self-righting capsule device, wherein when the self-righting capsule device is at least partially supported by the tissue of the lumen wall, the self-righting capsule device orients in a direction to allow the delivery member to be inserted into the lumen wall to deliver at least a portion of the therapeutic payload into the tissue.
